(19) United States
(12) Patent Application Publication
(10) Pub. No.: US 2018/0016631 A1
(43) Pub. Date: Jan. 18, 2018

(54) BACKBONE MEDIATED MATE PAIR SEQUENCING

(71) Applicant: Keygene N.V., Wageningen (NL)

(72) Inventor: Michael Josephus Theresia VAN EIJK, Wageningen (NL)

(73) Assignee: Keygene N.V., Wageningen, (NL)

(21) Appl. No.: 15/539,273

(22) PCT Filed: Dec. 23, 2015
(86) PCT No.: PCT/NL2015/050906

§ 371 Date: Jun. 23, 2017

(30) Foreign Application Priority Data
Dec. 24, 2014 (NL) 2014063

(51) Int. Cl. C12Q 1/68

(52) U.S. Cl. CPC C12Q 1/6874; C12Q 1/6806; C12Q 1/6855

(57) ABSTRACT

Disclosed is a method suitable for (long-range) mate pair sequencing wherein the mate pairs are located within a certain distance from each other on the same nucleotide sequence. By ligating a DNA fragment into an identifier section—containing backbone, a digestable circularized construct is provided to which adaptors can be ligated after digestion. Amplification yields amplicons that contain a combination of the identifier section with the terminal part of the fragments. The fragments are subsequently mated to each other to obtain a mated pair by identifying the corresponding identifier section in both amplicons. The mated pairs can be used in the construction of genome scaffolds or in the generation of draft genome sequences.

### FIELD OF THE INVENTION

The present invention relates to a method for the generation of mate pair sequences that may be used in the generation of (de novo) genome sequences. The invention relates in particular to the use of long-range mate pair sequencing to be applied in Whole Genome Sequencing.

### BACKGROUND OF THE INVENTION

Whole genome (re)sequencing is an important application of next generation sequencing technologies to create reference genomes as a tool to determine and understand genetic difference and to elucidate and better understand gene function. Various next generation sequencing platforms and genome sequencing approaches have been published and used to create draft and finished genome sequences. Current whole genome sequencing strategies involve the use of mate pair libraries of sample DNA to generate sequence reads that are used to create scaffolds that connect assembled sequence contigs. To this end, mate pair libraries are preferably made using large (1-15 kb) fragments, since longer fragments have a larger scaffolding potential. The current upper limit for mate-pair library construction is in the area of 10-15 kb.

Known solutions such as disclosed in WO2010/003316 are based on ligating size-selected, large insert DNA into modified Bacterial Artificial Clone (BAC) vectors that do not contain restriction sites, digesting the product with an restriction enzyme, re-circularizing the termini of the product, amplification of the re-ligated product and paired end sequencing of the amplicons. While these methods aim to increase the size limitation associated with current mate pair library preparation protocols (with upper limits of 10-15 kb as mentioned above) towards approximately 125 kb (i.e. the average insert size of typical BACs), these methods requires extensive modification of BAC vectors to eliminate restriction enzyme recognition sequences and incorporate amplification- and sequence primer binding sites. Moreover, transformation of the modified BAC vectors containing DNA insert into E. coli hosts is needed, combined with the need to use (modified) BAC vectors containing selection markers that are compatible with propagation and selection in E. coli hosts. Hence, current methods are in need of improvement to further enhance scope, reliability and simplicity of these methods. The present invention provides for these and other enhancements.

### SUMMARY OF THE INVENTION

The present inventor has found a method for the generation of mate pair sequences.

In one aspect, the invention pertains to a method for long-range (or long distance) mate pair sequencing wherein two sequences that are paired are determined. The two sequences are located within a certain distance from each other and are derived from the same nucleotide sequence/DNA fragment. By the provision of a DNA fragment and ligating it into a backbone that contains at least one identifier section and at least one primer binding site, a circularized fragment is provided. The circularized fragment is digested with a restriction enzyme to obtain a fragmented construct that contains the backbone and two partial fragments. By a combination of adaptor-ligation with primer binding site-containing adaptors and amplification, amplicons are obtained. For each fragmented construct, the amplicons contain a combination of the identifier section with one or both of the two partial fragments. Typically for each fragmented construct two amplicons are obtained wherein, typically, one amplicon contains at least one identifier section and one of the partial fragments and the other amplicon contains at least one identifier section and the other partial fragment. The partial fragments are subsequently mated to each other to obtain a mated pair by identifying the corresponding identifier section in both amplicons. The mated pairs can be used in the construction of genome scaffolds or in the generation of draft genome sequences.

### DESCRIPTION OF THE FIGURES

FIG. 1: a schematic overview of the method of the invention wherein a fragment (F) contains two terminal restriction fragments (F1,F2) which independently may have staggered (St) or blunt ends (BI). Backbones are provided which may be of two types (B1,B2). The backbone, which can be single stranded or double stranded, may have (when double stranded) staggered (St) and/or blunt ends (BI). B1 has a structure wherein two primer binding sites (PBS1, PBS2) are interspersed with an identifier section (ID), i.e. the identifier section (ID) is located between and may even be flanked by the two primer binding sites (PBS1, PBS2). B2 has a structure wherein a primer binding site (PBS) is located between two identifier sections (ID1, ID2). The identifier sections (ID, ID1, ID2) comprise a structure Nx, wherein N indicates the nucleotides of the identifier (or barcode), which is three or four nucleotides selected from the group consisting of A,C, T, and G and x is an integer indicating the number of nucleotides in the identifier. The number of nucleotides, x, is in one embodiment between 5 and 30, thus 5<x<30, preferably 10<x<20. Thus an identifier Nx is made up from the four nucleotides A, C,T, or G and preferably has a length of between 5 and 30 nucleotides. Thus, an alternative notation for an identifier is Nx=[A,C,T,G]₅₋₃₀ Alternatively the identifier uses only three out of the four nucleotides. Thus, an alternative notation for an identifier having from 10-20 nucleotides and composed of only A, T, or G is Nx=[A,T,G]₁₀₋₂₀. The two primer binding sites (PBS1, PBS2) may or may not be the same. The fragment (F) and the backbone (B1 or B2) are ligated to provide a circularized construct (C) having the structure F1-PBS1-ID-PBS2-F2 or F1-ID1-PBS-ID2-F2, wherein the underlining symbolises the circular structure as depicted in the figure.

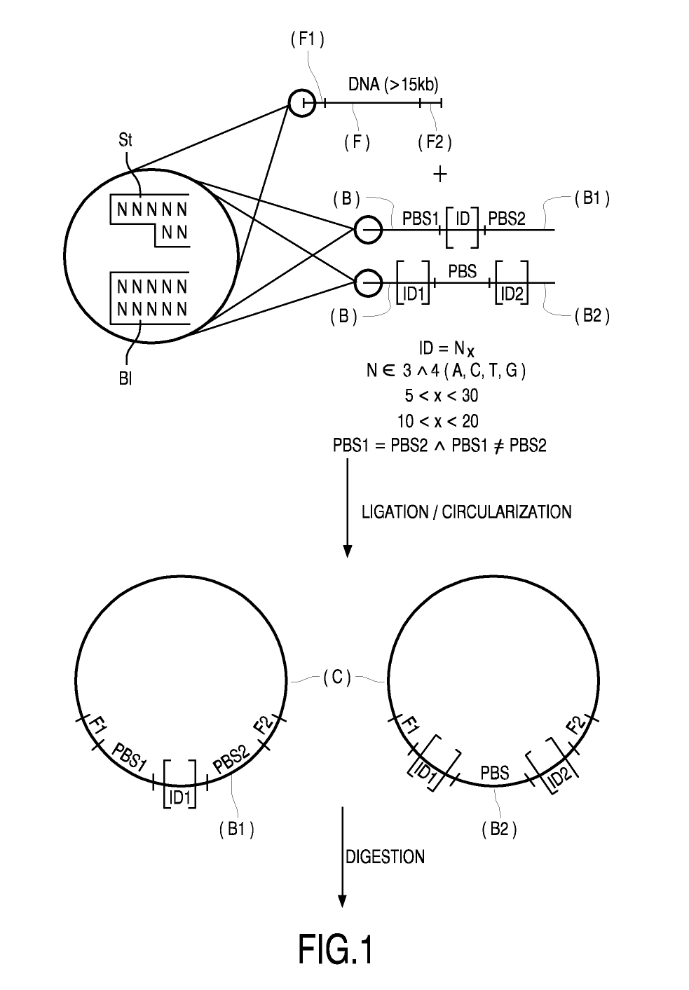

The circularised fragments are digested to yield a fragmented construct F1-PBS1-ID-PBS2-F2 (B1F) or F1-ID1-PBS-ID2-F2 (B2F). B1F or B2F can be independently blunt and/or staggered on either side but there is a preference for both ends having the same structure (blunt or staggered) (B1FSt, B2FSt, B1FBI, B2FBI). To these fragmented constructs adaptors are ligated (single stranded, double stranded blunt, double stranded staggered, Y-shaped blunt, Y shaped staggered). Possible combinations are listed in Table 1.

FIG. 2: schematic representation of the preferred combinations of fragmented constructs and adaptors. The preferred combinations are DStB1FSDSt, DStB2FSDSt, YStB1FSYSt, YStB2FSYSt, i.e. using staggered double stranded or Y-shaped adaptors.

FIG. 3: schematic representation of the use of intermediate adaptors (IA) when ligating a fragment into a backbone. The intermediate adaptors may have on either side a blunt or a staggered end, depending on the structure of the end of the fragment and the backbone.

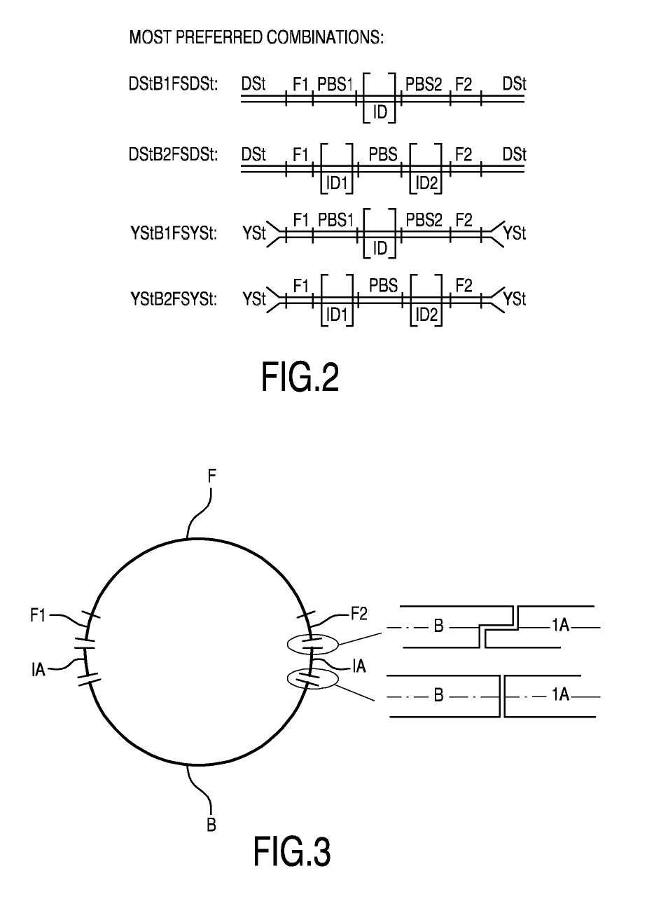

FIG. 4: schematic representation of the generation of a mated pair based on the identifier sections (ID, ID1, ID2), linking (mating) the two partial fragments (F1, F2). When a backbone of type B1 is used, the amplicons A1, A2 will contain the same identifier section (ID) (as identified in the sequence read) which mates F1 with F2. When a backbone of type B2 is used, Amplicon 1 (A1) contains ID1 and Amplicon 2 contains ID2. Retrieval of ID1 and ID2 from the sequence reads will provide the sequence of F1 and F2 respectively which are subsequently linked to form a mated pair (F1-F2).

### DETAILED DESCRIPTION OF THE INVENTION

The invention pertains to a method for mate-pair sequencing comprising the steps of
a. providing a DNA fragment (F);
b. providing an backbone (B), the backbone comprising one identifier section (ID) and at least one (first) primer binding site (PBS);
c. ligating both ends of the fragment (F) with the backbone (B), thereby circularizing the backbone to obtain a circularized construct (C);
d. digesting the circularized construct (C) with at least one enzyme (E) to obtain a fragmented construct comprising the backbone (B) and a first (F1) and a second (F2) partial fragment of the DNA fragment;
e. ligating adaptors (Ad) containing at least one (second) primer binding site (PBS) to the fragmented construct to obtain an adaptor-ligated fragmented construct;
f. amplifying the adaptor-ligated fragmented construct using one or more primers (P), thereby providing a first amplicon (A1) comprising the identifier section (ID) and the first partial fragment (F1) and a second amplicon (A2) comprising the identifier section (ID) and the second partial fragment (F2);
g. sequencing the amplicons (A1, A2) to determine of each amplicon the nucleotide sequence of the identifier section (ID) of the backbone and at least part of the partial fragment (F1,F2);
h. mating the first (F1) and second (F2) partial fragments based on the presence of the identifier section (ID) in the amplicons (A1, A2), thereby identifying the mated first (F1) and second (F2) fragment of the DNA fragment.

In the method of the present invention, a fragment (nucleic acid sequence) is provided as well as a backbone. The backbone contains a primer binding sequence and an identifier section. The fragment and the backbone are ligated to each other, thereby generating a circularized construct. In the circularized construct, the two ends of the fragment and the two ends of the backbone are connected to each other. The circularized construct is now digested with a restriction enzyme into parts (a fragmented construct). One of the parts of the circularised construct contains the backbone with on each side of the backbone a part of the fragment (partial fragment, F1, F2)). To these partial fragments, adaptors are ligated that each contain a primer binding sequence. The adaptor-ligated fragmented construct is now amplified using primers. One of the primers is directed towards a primer binding sequence in the backbone and the other primer is directed to a primer binding sequence in the adaptor. The amplification yields amplicons. Each amplicon contains an identifier section and one of the partial fragments (F1 or F2). Sequencing of the amplicons reveals the identifier section (or at least the identifier Nx in the identifier section, optionally combined with a sample-specific identifier also comprised in the identifier section or in a separate section of the backbone) and the partial fragment. By mating the identifier sections that are derived from the same backbone, the partial fragments are mated and a mated pair is obtained. Such a mated pair can be used for a variety of proposes such as in the generation, expansion or completion of sequence scaffolds and/or the completion of genome sequences, linking contigs from physical maps and so on.

Moreover, the present invention avoids the transformation of modified BAC vectors containing DNA insert into E. coli hosts and provides an in vitro methodology as opposed to an in vivo methodology without the need to use (modified) BAC vectors containing selection markers that are compatible with propagation and selection in E. coli hosts. Furthermore, the mate pair libraries of the present invention are not even limited in distance between the mates to the average of 125 kb typical for BAC libraries, but only limited to the size of the target DNA molecules from which mate pair sequences are needed.

The principle of the invention thus resides in the combination of one or more identifier sections in the same backbone with two partial fragments derived from a larger fragment wherein the one or more identifier section(s) serve(s) to link the partial fragments to the larger fragment and thereby generate a mated pair.

This generic principle can be embodied in a wide variety of embodiments and variants as will become clear herein below. Some variants and embodiments are focussed on a specific technical feature and are only described within the realms of that feature and not necessarily described directly in relation to all other embodiments, variations and permutations described herein. Nevertheless, it will be clear to the skilled person that, without it being explicitly mentioned, an embodiment, variant or permutation may and will find analogous application in other embodiments, without describing the whole method again. For instance variation in adaptors can be combined with variations in backbones without that combination being explicitly described other than through the dependency of the claims.

The DNA fragment (for instance a fragment of a nucleic acid sequence) is preferably obtained from a sample. The sample may be a DNA sample (S) comprising one or more selected from the group consisting of genomic DNA, genomic DNA from isolated chromosomes, genomic DNA from isolated chromosome regions, mitochondrial DNA, chloroplast DNA, viral DNA, microbial DNA, plastid DNA, synthetic DNA, DNA products of DNA amplifications, and cDNA.

The fragment may be obtained by digestion of one or more of the nucleic acids in the sample with an (restriction) enzyme. Thus, the nucleic acid sample may contain (a) restriction enzyme digestion site(s). The presence of a restriction enzyme digestion site is possibly known from the available sequence information, but it may also be derivable from statistical analysis/knowledge of the genome under investigation. Since restriction enzyme recognition sequences typically are 4-8 nucleotides long, the statistical occurrence of a recognition site will be, on average, every 256 nucleotides for a 4 bp cutter such as Msel. Such a digestion may be a partial digestion, i.e. the digestion with the restriction enzyme is performed for a period too short and/or a concentration of the enzyme that is deliberately too low for all restriction sites to be cut with the enzyme during the incubation period. The restriction enzyme may have a 3-5 bp recognition sequence (frequent cutter) or may be have a 6-8 bp recognition sequence (rare cutter). The fragment may also be provided by a combination of two or more rare and/or frequent cutters. The fragments may also be provided by application of mechanical force and/or by random fragmentation, preferably selected from the group consisting of shearing, sonication, and nebulization of the DNA sample. The length distribution of the fragments may vary with the intensity of the fragmentation process. The selection of the combination of restriction enzymes and/or mechanical force based fragmentation techniques may depend on the (range of the) desired fragment size and can be readily determined by the skilled person. The obtained fragment may have a staggered end and/or a blunt end, depending on the fragmentation technique. Fragments having staggered ends may be blunted by known techniques, such as with an enzyme, preferably an endonuclease, a flap endonuclease or a polymerase. The fragments may also be phosphorylated using known techniques. When the fragment contains a staggered end, the nucleotide sequence of the overhang may be known, for instance when a restriction enzyme is used that generates known ends (such as a class II restriction enzyme).

The fragment obtained from the sample can be size selected, for instance on a gel or using other common techniques for size selection. Although the method presented here is generic in the sense that it is independent of any species, prior sequence information or fragment size, it is preferred that a size selection is performed to yield a fragment that has a size of more than 15 kilobasepairs (kb), more than 25 kb, more than 50 kb, more than 75 kb, more than 100 kb, or more than 150 kb. With fragments in that range (i.e. above the mentioned fragment sizes), mated pairs can be generated that are adequate for long-range scaffold building purposes. Nevertheless, the same method can be used to generate mated pairs of shorter range that may be also used in the generation of the scaffold and the genome sequence. Thus in another embodiment, the fragment may be more than 1 kb, more than 5 kb or more than 10 kb or between ranges that are flanked by the abovementioned fragment length (such as between 10 kb and 25 kb, between 5 and 15 kb, between 5 and 50 kb and so on).

The backbone that is used in the present invention is a nucleotide sequence (oligonucleotide) that is preferably synthetic, i.e. chemically synthesised or composed of individual parts or sections that have been synthetically prepared, for instance on an array, wherein the parts may be enzymatically combined into the backbone. The length of the backbone may vary, but is typically in the range of 30-250 nucleotides. The length is primarily determined by the various functionalities that are incorporated in the backbone as described herein. A backbone may be single stranded or double stranded and may have blunt and/or staggered ends. In preferred embodiments, the backbone is free from (does not contain) recognition sites for a restriction enzyme that is used in the subsequent digesting step of the circularised fragment and/or is free of palindromic sequences of four bases or greater in length. The backbone contains one, two or more identifier sections. The identifier section in the backbone comprises a barcode N of x nucleotides (Nx). The identifier section serves to identify the fragments ligated into the backbone. The backbone and/or the identifier section may contain other functionalities such as a sample-specific identifier which may have a similar structure as the barcode. The barcode may also be composed of a sample-specific part and a fragment-specific part or the barcode may be designed such that each individual barcode is assigned to a fragment from a sample (i.e. using longer barcodes). The nucleotides N in the backbone can be selected from amongst all nucleotides preferably from amongst all four (A,C,T, G) or in certain embodiments, from amongst three out of A,C,T or G (so A,C,T; A,T,G; A,C,G; C,T,G). The latter embodiment would obviate or simplify the need for the backbone being free of recognition sequences for restriction enzymes. The number (x) of nucleotides in an identifier may vary widely, but is typically between four and fifty, preferably x is 5-30, preferably 10-20. A preferred type of identifier does not contain (is free of) two or more identical consecutive bases, as it reduces or prevents false readings due to read-throughs during sequencing with sequencing chemistries that are prone to homopolymer errors, i.e. have an elevated error rate in sequencing stretches of consecutive identical nucleotides.

The number of available unique identifiers and hence the number of backbones provided preferably exceeds the number of sequence reads produced in a typical sequence run.

In one embodiment of the backbone, the backbone contains one or more identifiers (ID), depending on the structure of the backbone. The identifier serves to identify the origin of the first and second fragment after the sequencing step. The identifier serves to link the first and second partial fragment (F1, F2) to each other as being derived from the same fragment (F). Partial fragments that originate for the same fragment are linked to that fragment by virtue of the one or more identifier(s) derived from the same backbone.

In one embodiment, the backbone contains an identifier (ID) located in between two primer binding sites. In another embodiment, the backbone contains a primer binding site located in between two identifier sections (ID1, ID2). Since the backbones are artificially and designed, ID1 may be same or may be different from ID2. In the latter case, for proper designation of sequence reads to be mates, it is preferably known which combination of ID1 and ID2 are part of the same backbone molecule.

Thus, the invention also pertains to a method for mate-pair sequencing comprising the steps of:

a. providing a DNA fragment (F);
b. providing an backbone (B), the backbone comprising two identifier sections (ID1, ID2) and wherein at least one (first) primer binding site (PBS) is preferably located in between the two identifier sections (ID1, ID2);
c. ligating both ends of the fragment (F) with the backbone (B), thereby circularizing the backbone to obtain a circularized construct (C);
d. digesting the circularized construct (C) with at least one enzyme (E) to obtain a fragmented construct comprising the backbone (B) and a first (F1) and a second (F2) partial fragment of the DNA fragment;
e. ligating adaptors (Ad) containing at least one (second) primer binding site (PBS) to the fragmented construct to obtain an adaptor-ligated fragmented construct;
f. amplifying the adaptor-ligated fragmented construct using one or more primers (P), thereby providing provides a first amplicon (A1) comprising one of the two identifier sections (ID1) and the first partial fragment (F1) and a second amplicon (A2) comprising the other of the two identifier sections (ID2) and the second partial fragment (F2);
g. sequencing the amplicons (A1, A2) to determine of each amplicon the nucleotide sequence of the identifier section (ID1, ID2) of the backbone and at least part of the partial fragment (F1,F2);
h. mating the first (F1) and second (F2) partial fragments based on the presence of the identifier section (ID) in the amplicons (A1, A2), thereby identifying the mated first (F1) and second (F2) fragment of the DNA fragment.

Methodologies for generating libraries of backbones containing unique identifiers are known in the art, i.e. via (separate) randomised synthesis of Nx and subsequent incorporation in a generic backbone or via structured oligosynthesis, such as on an array, where deliberate and pre-designed libraries of backbones are build containing known and pre-designed sequences, including identifiers.

Either way, the backbone contains means of identification in the backbone by the presence of one or more identifiers such that the partial fragments that are obtained from the fragment are linked (‘mated’) to each other in the sense that it is known which first partial fragment occurs in the fragment together with which second partial fragment such that they can form a mated pair or a mate pair.

Libraries of identifiers can be used. Such libraries can be used to accommodate a multitude of fragments, for instance derived from a sample. Such a multitude of fragments can be two or more fragments and may also be more than 10, 100, 1000 or even 10 thousands of fragments, such as a set of fragments obtained from fragmenting a genome or a chromosome or a BAC library or part thereof, such as disclosed herein elsewhere. As stated elsewhere, the number of identifiers in a library preferably exceeds the number of fragments. The library can be obtained by technology known in the art as barcoded DNA or by building libraries of identifiers of certain length than contain permutations of nucleotide such that each identifier in the library is unique, i.e. occurs only once in the entire library. A library of identifiers of 15 nucleotides in length built from all four nucleotides can contain (4exp15) 1.07*10exp9 unique combinations. With the requirement that no two consecutive nucleotides are the same this number will be reduced, but the number of remaining unique identifiers is still adequate for most purposes. Thus, with the identifiers a library of backbones can be constructed, the backbones having a structure as outlined herein elsewhere with identifiers section(s) and primer binding site(s). Such a library can contain more than two distinct backbones (i.e. containing different identifiers), preferably more than 100, 1.000, 5.000 or even 10.000 backbones. Numbers higher than 10.000 are also feasible; in fact the length of the identifier is the only limitation and increasing the identifier length can be used to increase the complexity of the backbone library. The backbones in a library are designed (constructed) such that each identifier is unique in the library and preferably the backbone is unique within the library by virtue of the identifier in the backbone or by the combination of the identifiers in the backbone. Thus, each identifier section or combination of identifier sections in a backbone of the library is different from any other backbone comprising an identifier section or combination of identifier sections in the library of backbones. Each backbone in the library is unique in the library of backbones.

All identifiers in the library of backbones differ from each other by at least two nucleotides to enhance the discrimination between the identifiers and hence between the backbones in the library.

The fragment (F) is ligated with the backbone. The ligation circularizes the backbone with the fragment. The fragment hence ligates with both ends to both ends of the backbone, thereby providing a circularized construct (C). The conditions for circularizing the fragment with the backbone are well understood and can be applied using conventional techniques in the art

The term “ligation” refers to the enzymatic reaction catalyzed by a ligase enzyme in which two (double-stranded) DNA molecules are covalently joined together. In general, for double stranded DNA strands, both DNA strands are covalently joined together, but it is also possible to prevent the ligation of one of the two strands through chemical or enzymatic modification(s) of one of the ends of the strands. In that case the covalent joining will occur in only one of the two DNA strands.

The term “ligating” refers to the process of joining separate (double) stranded nucleotide sequences. The double stranded DNA molecules may be blunt ended, or may have compatible overhangs (sticky overhangs) such that the overhangs can hybridize with each other. Alternatively, one of the DNA molecules may be double stranded with an overhang to which overhang another single stranded DNA molecule (single stranded adaptor) can anneal. The joining of the DNA fragments may be enzymatic, with a ligase enzyme, DNA ligase. However, a non-enzymatic, i.e. chemical ligation may also be used, as long as DNA fragments are joined, i.e. forming a covalent bond. Typically a phosphodiester bond between the hydroxyl and phosphate group of the separate strands is formed in a ligation reaction. Double stranded nucleotide sequences may have to be phosphorylated prior to ligation.

The fragment may be blunt and/or staggered on one or on both ends and the backbone can be designed accordingly. For instance for staggered ends of fragments, the use of backbones having a staggered end, and for blunt ends of fragments, the use of backbones having a blunt end can be used. In case multiple fragments are ligated into backbones of which fragments the ends independently can be staggered or blunt, the library of backbones may also contain backbones that have blunt and/or staggered ends.

The fragments may be ligated with intermediate adaptors and subsequently or simultaneously be ligated into the backbone. These adaptors function as intermediate adaptors prior to the circularization of the fragment and the backbone. The use of intermediate adaptors may be advantageous if one or both of the ends of the fragment are not known or are blunt(ed), due to the way the fragment is obtained (for instance via random fragmentation). The intermediate adaptors then may be blunt on one end for ligation with the end of the fragment and staggered on the other end, or instance being specific for one of the ends of the (staggered) backbone. Alternatively, the intermediate adaptor (or a set thereof) may be specific for the backbone on one end and contain an overhang on the other end that contains a permutation of the overhanging nucleotides to accommodate all possible staggered ends of fragment. This could be particularly practical when using multiple fragments obtained via a technique that provides staggered ends of unknown or at least varying sequence and a library of backbones.

Thus, in certain embodiments, the fragment is ligated with a first and/or a second (intermediate) adaptor prior to (or simultaneous with) ligation into the backbone. The adaptor can have a first end to be ligated to the backbone and a second end to be ligated to the fragment. In certain embodiments, the backbone has one or two staggered ends and the first end of the adaptor is staggered to be selectively ligated to the backbone. In certain embodiments, the backbone has a first and a second end which are both staggered and the first and a second staggered ends have a different sequence overhang. In certain embodiments, two adaptors are provided having first ends that each can be selectively ligated to the first and second end of the backbone, respectively. In certain embodiments, the second end of the first and/or the second adaptor is blunt, to be ligated to a blunt fragment. In certain embodiments, a set of (intermediate) adaptors is provided, each containing on the second end of the adaptor a permutated overhang to be ligated to staggered fragments.

Alternatively, a library of backbones may be provided that at their ends contain permutated overhangs, i.e. all possible combinations of nucleotides.

The intermediate adaptors used in the present invention, can have a length of from 8-100 bp, preferably from 10-25 bp.

As used herein, the term “adaptors” or intermediate adaptors refers to short, typically double-stranded, DNA molecules with a limited number of base pairs, e.g. about 10 to about 30 base pairs in length, which are designed such that they can be ligated to the ends of (restriction) fragments. Double stranded adaptors are generally composed of two synthetic oligonucleotides that have nucleotide sequences which are partially complementary to each other. An adaptor may have blunt ends, or may have staggered ends, or may have a blunt end and a staggered end. A staggered end is a 3′ or 5′ overhang. When mixing the two synthetic oligonucleotides in solution under appropriate conditions, they will anneal to each other forming a double-stranded structure. Adaptors can also be single stranded, in which case it may be convenient and preferred if one of the ends of the single stranded adaptor is compatible for at least a few nucleotides (2, 3, 4 or 5) with one of the strands of one of the ends of a (restriction) fragment, such that the singe stranded adaptors are capable of annealing to the (restriction) fragment. To that end a fragment may be extended by the addition of nucleotides to one of the ends of the fragment. One end of the adaptor molecule can be designed such that, after annealing, it is compatible with the end of a (restriction) fragment and can be ligated thereto. The other end of the adaptor (either in the single strand version or in the double strand version) can be designed so that it cannot be ligated (i.e. blocked). This allow for only one end of the adapter to be ligated or for only one of the strands of a double stranded adapter to be ligated. However, when an adaptor is to be ligated in between DNA fragments (intermediate adaptor), both ends of one of the strands of the adaptor are ligatable. Being ligatable in general implies the presence of 3′-hydroxyl or 5′-phosphate groups. Being blocked from ligation generally means that the required 3′ and 5′ functionalities are lacking or blocked. In certain cases, adaptors can be ligated to fragments to provide for a starting point for subsequent manipulation of the adaptor-ligated fragment, for instance for amplification or sequencing. In the latter case, so-called sequencing adaptors may be ligated to the fragments. Being compatible for ligation can be accomplished in two (combined) ways: the end of the (double-stranded) adaptor contains an (overhanging) section that is compatible with the overhanging end of a restriction fragment such that the adaptor and the fragment may anneal. A second way is that the nucleotide that is located at the end of one strand of the adaptor is provided in such a way that it can chemically be coupled to another nucleotide, for instance from a restriction fragment. Alternatively, a nucleotide at the end of an adaptor can also be modified (blocked) such that it cannot be coupled to another nucleotide. Double stranded adaptors may have these features combined such that the double stranded adaptor is capable of annealing to a fragment and one or both strands can be coupled to the fragment. The adaptor (whether double or single stranded) is ligated to the end of the (restriction) fragment using a ligase. The result is an adaptor-ligated (restriction) fragment. In one embodiment, the ligation of the at least one adaptor occurs at the 5′end of the (restriction enzyme digested) fragment(s). In one embodiment, the ligation of the at least one adaptor occurs at the 3′ end of the (restriction enzyme digested) fragment(s).

As an alternative to adaptor-ligation (whether single or double stranded), nucleotides may be added to the fragments, preferably at their 3′-end using commonly known nucleotide extension methods thereby introducing, preferably in a known order, an elongation of the fragment with a known sequence (a nucleotide elongated sequence), for instance by a sequence of steps each time introducing one nucleotide at a time (single nucleotide extension) to thereby elongate fragments with 3-100 nucleotides, preferably with 5-50 nucleotides and with higher preference with 18-40 nucleotides, with 10-20 nucleotides being most preferred. This elongation of fragments results in nucleotide-elongated fragments.

Thus, the fragment is ligated into the backbone with or without the use of intermediate adaptors on one or both ends to provide circularized constructs of the fragment.

The backbone may further contain an affinity tag (such as biotin) to remove the backbone from the reaction mixture. The non-circularized fragments and/or backbones may be removed. Also, the non-circularized fragments may be removed by an exonuclease treatment or another treatment to remove all linear DNA from the mixture. Alternatively, the backbones may be removed from the mixture using the affinity tag or a combination of both methods may be used. Also a capturing probe may be used on the circularized fragments or on the non-circularized fragments.

In a further step, the circularized construct can be digested with an enzyme (E), preferably with at least one restriction enzyme, to provide a fragmented construct that comprises the backbone (B), and a first (F1) and a second (F2) partial fragment of the DNA fragment (F).

Thus the digestion of the circularized construct with the enzyme provides a set of fragments, one of which will contain the backbone (the fragmented construct). Since the backbone is typically constructed or designed such that the backbone remained unaffected by the enzyme (for instance due to the absence of a recognition sequence of the enzyme used), there is one fragment that contains the backbone and on either end of the backbone a part of the fragment, i.e. the terminal ends of the fragment. These ends are indicated as the partial fragment (F1, F2). In one embodiment, wherein the backbone contains two identifiers as outlined herein elsewhere, the backbone may contain a recognition sequence for a restriction enzyme located between the two identifiers. Preferably the backbone then also contains two primer binding sites such that the principal structure is ID-PBS-REsite-PBS-ID. Upon circularization of the construct with such a backbone, the IDs are linked and so are their partial fragments (F1, F2) even if their subsequent separation due to the digestion renders them individual. The partial fragments (F1,F2) can each independently have a length of preferably between 30 and 20,000 bp, more preferably between 30 and 5,000 bp and even more preferably between 30 and 500 bp.

The enzyme is preferably a restriction enzyme. As used herein, the term “restriction enzyme” or “restriction endonuclease” (the terms ‘restriction enzyme’ and ‘restriction endonuclease’ are used interchangeably) refers to an enzyme that recognizes a specific nucleotide sequence (recognition site) in a double-stranded DNA molecule, and will cleave both strands of the DNA molecule at or near every recognition site, leaving a blunt or a staggered end. Also encompassed are so-called nicking restriction enzymes that contain recognition sites for single or double strand DNA but subsequently cut (nick) in only one strand.

As used herein, the term “isoschizomers” refers to pairs of restriction enzymes which are specific to the same recognition sequence and which cut in the same location. For example, Sph I (GCATĜC) and Bbu I (GCATĜC) are isoschizomers of each other. The first enzyme to recognize and cut a given sequence is known as the prototype, all subsequent enzymes that recognize and cut that sequence are isoschizomers. An enzyme that recognizes the same sequence but cuts it differently is a neoschizomer. Isoschizomers are a specific type (subset) of neoschizomers. For example, Sma I (CCĈGGG) and Xma I (ĈCCGGG) are neoschizomers (not isoschizomers) of each other. Isoschizomers and neoschizomers can be used in the present invention. The same description may apply to the restriction enzymes that may be used in providing the fragment from the DNA sample and that may be used in the digestion of the circularized fragment.

The term “Class-II restriction endonuclease” refers to an endonuclease that has a recognition sequence that is located at the same location as the restriction site. In other words, Class II restriction endonucleases cleave within their recognition sequence. Examples thereof are EcoRI (G/AATTC) and Small (CCC/GGG).

The term “Class-IIS restriction endonuclease” refers to an endonuclease that has a recognition sequence that is distant from the restriction site. In other words, Class IIS restriction endonucleases cleave outside of their recognition sequence to one side.

Examples thereof are NmeAIII (GCCGAG(21/19), FokI (GGATG9/13), and AlwI (GGATC4/5). A “Class-IIB restriction endonuclease” refers to an endonuclease that has a recognition sequence that is distant from the restriction site and wherein there are two restriction sites, located on both sides of the recognition sequence. In other words, Class IIB restriction endonucleases cleave outside of their recognition sequence at both sides.

The restriction enzyme can be any restriction enzyme such as one that has 3-5 bp recognition sequence (frequent cutter) or a 6-8 bp recognition sequence (rare cutter). The fragments of the circularised construct are preferably obtained by restricting the circularized construct with a combination of one or more frequent and/or rare cutters. The restriction enzyme can be of a variety of types with a preference for Class II, IIB, and IIS, more preferably Class II.

The fragments that do not contain the backbone can be removed from the mixture or separated form the non-backbone containing fragments, for instance by a size separation step and subsequent isolation of the fraction that contains the fragmented construct composing the backbone or by using an affinity tag such as biotin, preferably in the backbone, as explained herein before.

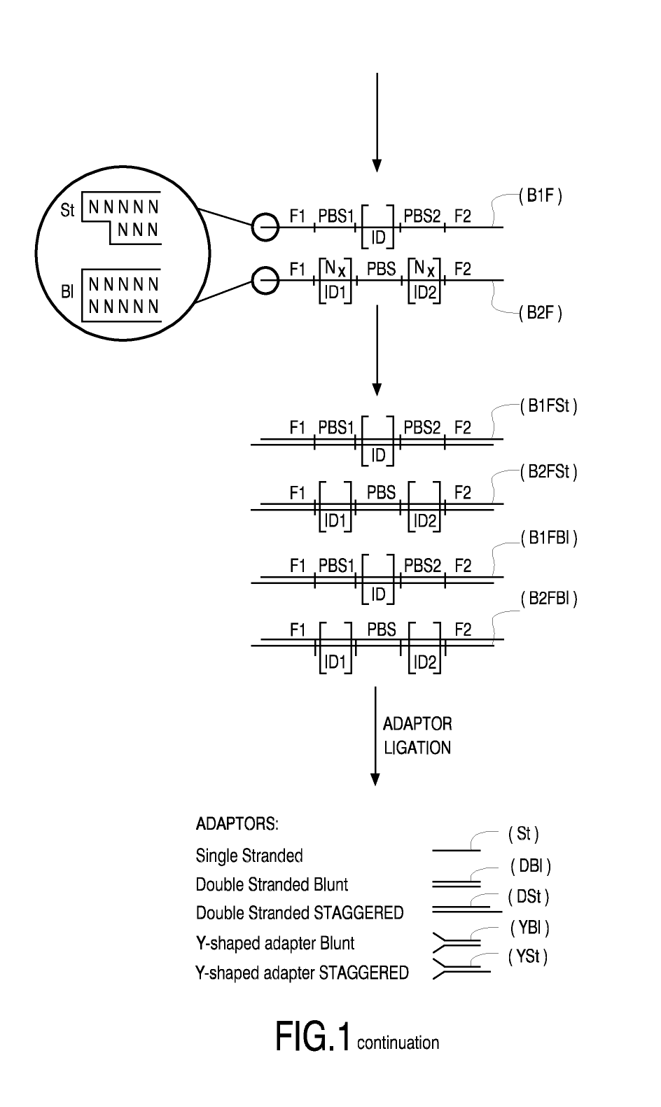

To the fragmented construct (i.e. the backbone-containing fragment of the circularized construct obtained after fragmentation) adaptors are ligated. Adaptors are defined also herein elsewhere. One or more adaptors (Ad) can be ligated to one or both ends of the fragmented constructs. The adaptors may be the same or different. The adaptor contains a primer binding site (PBS). The result of the adaptor ligation to the fragmented construct is an adaptor-ligated fragmented construct. The adaptor itself can have a variety of structures so that the adaptor is selected from the group consisting of a single stranded adaptor (S), a double stranded adaptor (D), and a Y-shaped adaptor (Y). A double stranded or a Y-shaped adaptor may have a blunt (BI) or a staggered (St) end, depending on the structure of the free end of the partial fragment. For each end of the fragmented construct another adaptor can be designed and/or selected. Thus, two adaptors (Ad1, Ad2) can be ligated, one to each end of the fragmented construct, that are independently selected from a single stranded (S), double stranded (D) or Y shaped adaptor (Y). In case of a Y-shaped adaptor, at least one of the arms (Y1, Y2) of the Y-shaped adaptor contains a primer binding site (PBS). See Table 1 for combinations of backbones and adaptors. Preferred adaptor-ligated fragmented constructs are depicted in FIG. 2.

In certain embodiments, the fragmenting (for instance by digestion with a restriction enzyme) of the circularized construct and the ligation of adaptors can be performed simultaneously. In such an embodiment, it is preferred that the ligation of an adaptor does not restore the recognition sequence (RS) of the restriction enzyme (E).

The adaptors that are ligated to the fragmented construct and in particular to the ends of the partial fragments (F1, F2) contain primer binding sites, resulting in adaptor-ligated fragmented constructs containing primer binding sites both in the adaptors and in the backbone (commonly indicated as PBS, individually indicated as PBS1,PBS2, PBS3, PBS4).

The primer binding sites (PBS1,PBS2, PBS3, PBS4) in the adaptor-ligated fragmented construct may be the same or different and consequently one, two, three or four primers can be used in the amplification step. Thus, in certain embodiments, the one or two primer binding sites (PBS1, PBS2) in the backbone and the primer binding sites (PBS3, PBS4) in the adaptors are identical (PBS1=PBS2=PBS3=PBS4) and the adaptor-ligated construct is amplified from one primer (P1). In another embodiment, the backbone contains two identical primer binding sites (PBS1, PBS2; PBS1=PBS2) and the adaptors contain two identical primer binding sites (PBS3, PBS4; PBS3=PBS4) and the adaptor-ligated construct is amplified from two primers (P1, P2). In yet another embodiment, the backbone contains two identical primer binding sites (PBS1, PBS2; PBS1=PBS2) and the adaptors contain two different primer binding sites (PBS3, PBS4; PBS3≠PBS4), or the adaptors contain two identical primer binding sites (PBS3, PBS4; PBS3=PBS4) and the backbone contains two different primer binding sites (PBS1, PBS2; PBS1≠PBS2), and the adaptor-ligated construct is amplified from three primers (P1, P2, P3). In another embodiment, the backbone contains two different primer binding sites (PBS1, PBS2; PBS1≠PBS2) and the adaptors contain two different primer binding sites (PBS3, PBS4; PBS3≠PBS4) and the adaptor-ligated construct is amplified from four primers (P1, P2, P3, P4).

The adaptor-ligated fragmented construct can be amplified using conventional methods for the amplification of nucleotide samples such as PCR or isothermal amplification methods. The result of the amplification is an amplicon (A). When the adaptor-ligated fragmented construct is in fact a plurality of adaptor-ligated fragmented constructs, for instance in case the method of the invention used a plurality of fragments, such as from a DNA sample that was fragmented after which the fragments have been ligated into a backbone library, the amplification can be performed on the entire set (plurality) of adaptor-ligated fragmented constructs or the adaptor-ligated fragmented constructs can be split in two or more subsamples and separately amplified using different combinations of primers.

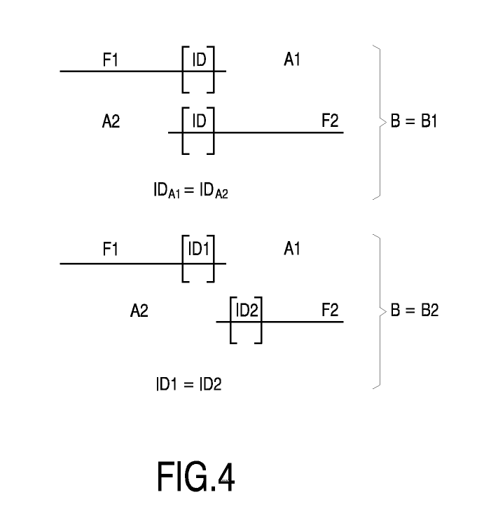

In certain embodiments, when the backbone contains two identifier sections (a first identifier section (ID1) and a second identifier section (ID2), the first amplicon (A1) contains the first identifier section (ID1) and the first partial fragment (F1) and the second amplicon (A2) contains the second identifier section (ID2) and the second partial fragment (F2) (see FIG. 4).

The amplicons are sequenced, preferably using high throughput sequencing such as Illumina's Sequencing by Synthesis platforms or by 454 sequencing technologies from Roche (GSII or GS FLX) or sequencing technologies such as generically indicated as Next-Next generation sequencing and/or SMRT sequencing (Pacific Biosciences (PacBio) etc. and described inter alia in Quail et al. BMC Genomics 2012, 13:341, to provide sequenced amplicons. Thus, the terms “high throughput sequencing” and “next generation sequencing” refer to sequencing technologies that are capable of generating a large amount of sequence reads, typically in the order of many thousands (i.e. ten or hundreds of thousands) or millions of sequence reads rather than a few hundred at a time. High throughput sequencing is distinguished over and distinct from conventional Sanger or capillary sequencing.

Typically, the sequenced products of high through put sequencing have relative short reads, between about 30 and 300 bases. Examples of such methods are given by the pyrosequencing-based methods disclosed in WO 03/004690, WO 03/054142, WO 2004/069849, WO 2004/070005, WO 2004/070007, WO 2005/003375, and by Seo et al. (2004) Proc. Natl. Acad. Sci. USA 101:5488-93. Currently, the PacBio RS platform produces read lengths up to 20 kb. These technologies further comprise extensive and elaborate data storage and processing workflows for read assembly etc. The availability of high throughput sequencing requires many conventional workflows and methods for the analysis of genomes to be redesigned to accommodate the type and quality of data that can be produced. Next generation high throughput sequencing is extensively described also in “Next Generation Genome sequencing” M. Janitz Ed. (Wiley-Blackwell, 2008).

Certain high throughput sequencing methods use amplification as an integral part of the method. In this respect it is noted that the step of amplification of adaptor-ligated fragmented constructs in the present method can be an integral part (i.e. combined or coincide with) the sequencing step and one or more of the primers used in the amplification is or contains a sequencing primer. A sequencing primer in this respect is a primer such as employed by or directly applicable to certain high throughput sequencing platforms and are provided or designed by the manufacturer. Examples thereof are P5 and P7 primers used in Illumina sequencing. The primers (in general, thus in a separate amplification as well as in an amplification as an integral part of the high throughput sequencing) may also contain an affinity probe such as biotin.

The sequenced amplicons that are provided by the invention contain the sequence information of the first partial fragment (F1) with the identifier (ID) or contain the sequence information of the second partial fragment (F2) with the identifier (ID). Thus they share the identifier sequence (ID). Or, in the embodiment wherein there are two identifiers (ID1, ID2) present in the backbone, the amplicons contains the sequence information of F1 combined with one of ID1 or ID2 and of F2 combined with the other of ID1 or ID2. The shared presence of the ID (or combined presence of ID1, ID2 for that matter) then links or mates the sequences of F1 and F2 together such that they become a mated pair (F1-F2). For F1 and F2 it is then known that they are derived from the same fragment, regardless of the distance between them in the DNA sequence that is under investigation. Thus, the mating of the first and second partial fragments is based on the presence of identical identifier sections (ID) in the amplicons (or based on linked first and second identifier sections ID1, ID2).

In embodiments of the invention, a plurality of samples can be analysed (i.e. two or more). To distinguishes between samples further identifiers can be used, incorporated in the backbone. This can be achieved by incorporating separate identifiers in the (library of) backbone(s) that is used for each sample. In this embodiment, the sequencing step may then incorporate also the sequencing of the sample specific identifier. Also the already present identifier section (ID, ID1, ID2) can contain a sample specific part.

The mated pairs obtained by the method of the present invention can be used in building a genome scaffold, or by complementing a physical map by further linking existing contigs. One of the technical advantages of the present invention is that it reduces PCR amplicon size compared to conventional BAC vector backbones and hence can lead to a higher library coverage and a more even amplification. Furthermore the method is advantageous in that that since both termini (F1, F2) are amplified separately, the presence of two and no more than two occurrences of the shared or combined identifier is indicative of a mated pair.

TABLE 1
Combinations of Backbones (B1, B2) with fragmented constructs (F) having on
either side partial fragments (F1, F2) having blunt (Bl) or staggered (St) ends and adaptors
(S, DBl, DSt, YBl, YSt) that are capable of ligating to the partial fragments:

| F1 | Fragmented Construct | | | | F2 |
|---|---|---|---|---|---|
| side | _B1FSt_{—} | _B2FSt_{—} | _B1FBl_{—} | _B2FBl_{—} | side |
| S | S_B1FSt_S | S_B2FSt_S | S_B1FBl_S | S_B2FBl_S | S |
| DBl | DBl_B1FSt_S | DBl_B2FSt_S | DBl_B1FBl_S | DBl_B2FBl_S | S |
| DSt | DSt_B1FSt_S | DSt_B2FSt_S | DSt_B1FBl_S | DSt_B2FBl_S | S |
| YBl | YBl_B1FSt_S | YBl_B2FSt_S | YBl_B1FBl_S | YBl_B2FBl_S | S |
| YSt | YSt_B1FSt_S | YSt_B2FSt_S | YSt_B1FBl_S | YSt_B2FBl_S | S |
| S | S_B1FSt_DBl | S_B2FSt_DBl | S_B1FBl_DBl | S_B2FBl_DBl | DBl |
| DBl | DBl_B1FSt_DBl | DBl_B2FSt_DBl | DBl_B1FBl_DBl | DBl_B2FBl_DBl | DBl |
| DSt | DSt_B1FSt_DBl | DSt_B2FSt_DBl | DSt_B1FBl_DBl | DSt_B2FBl_DBl | DBl |
| YBl | YBl_B1FSt_DBl | YBl_B2FSt_DBl | YBl_B1FBl_DBl | YBl_B2FBl_DBl | DBl |
| YSt | YSt_B1FSt_DBl | YSt_B2FSt_DBl | YSt_B1FBl_DBl | YSt_B2FBl_DBl | DBl |
| S | S_B1FSt_DSt | S_B2FSt_DSt | S_B1FBl_DSt | S_B2FBl_DSt | DSt |
| DBl | DBl_B1FSt_DSt | DBl_B2FSt_DSt | DBl_B1FBl_DSt | DBl_B2FBl_DSt | DSt |
| DSt | DSt_B1FSt_DSt | DSt_B2FSt_DSt | DSt_B1FBl_DSt | DSt_B2FBl_DSt | DSt |
| YBl | YBl_B1FSt_DSt | YBl_B2FSt_DSt | YBl_B1FBl_DSt | YBl_B2FBl_DSt | DSt |
| YSt | YSt_B1FSt_DSt | YSt_B2FSt_DSt | YSt_B1FBl_DSt | YSt_B2FBl_DSt | DSt |
| S | S_B1FSt_YBl | S_B2FSt_YBl | S_B1FBl_YBl | S_B2FBl_YBl | YBl |
| DBl | DBl_B1FSt_YBl | DBl_B2FSt_YBl | DBl_B1FBl_YBl | DBl_B2FBl_YBl | YBl |
| DSt | DSt_B1FSt_YBl | DSt_B2FSt_YBl | DSt_B1FBl_YBl | DSt_B2FBl_YBl | YBl |
| YBl | YBl_B1FSt_YBl | YBl_B2FSt_YBl | YBl_B1FBl_YBl | YBl_B2FBl_YBl | YBl |
| YSt | YSt_B1FSt_YBl | YSt_B2FSt_YBl | YSt_B1FBl_YBl | YSt_B2FBl_YBl | YBl |
| S | S_B1FSt_YSt | S_B2FSt_YSt | S_B1FBl_YSt | S_B2FBl_YSt | YSt |
| DBl | DBl_B1FSt_YSt | DBl_B2FSt_YSt | DBl_B1FBl_YSt | DBl_B2FBl_YSt | YSt |
| DSt | DSt_B1FSt_YSt | DSt_B2FSt_YSt | DSt_B1FBl_YSt | DSt_B2FBl_YSt | YSt |
| YBl | YBl_B1FSt_YSt | YBl_B2FSt_YSt | YBl_B1FBl_YSt | YBl_B2FBl_YSt | YSt |
| YSt | YSt_B1FSt_YSt | YSt_B2FSt_YSt | YSt_B1FBl_YSt | YSt_B2FBl_YSt | YSt |

### LIST OF ABBREVIATIONS

F: Fragment (of a nucleic acid sample)
F1, F2, . . . : partial fragments of F
B, B1, B2 . . . : Backbone
PBS, PBS1, PBS2, . . . : primer binding sequence, a nucleic acid section that is designed to pair with a primer
ID, ID1, ID2 . . . : Identifier
[Nx]: An Identifier or barcode in a Backbone comprising x nucleotides
x: integer (1, 2, 3, . . . )
C: circularized construct
E: (restriction) enzyme
BI: Blunt-ended
St: Staggered-ended
Ad, Ad1, Ad2: Adaptor
Ds or D: Double Stranded Adaptor
S: Single stranded Adaptor
Ys or Y: Y-shaped Adaptor
Pr, Pr1, Pr2, . . . : Primer
A, A1, A2, . . . : amplicon
IA: Intermediate adaptor

1.-75. (canceled)

76. A method for mate-pair sequencing comprising the steps of
a. providing a DNA fragment;
b. providing an backbone, the backbone comprising one identifier section and at least one first primer binding site;
c. ligating both ends of the DNA fragment with the backbone, thereby circularizing the backbone to obtain a circularized construct;
d. digesting the circularized construct with at least one enzyme to obtain a fragmented construct comprising the backbone and a first and a second partial fragment of the DNA fragment;
e. ligating adaptors containing at least one second primer binding site to the fragmented construct to obtain an adaptor-ligated fragmented construct;
f. amplifying the adaptor-ligated fragmented construct using one or more primers, thereby providing a first amplicon comprising the identifier section and the first partial fragment and a second amplicon comprising the identifier section and the second partial fragment;
g. sequencing the first and second amplicons to determine of each amplicon the nucleotide sequence of the identifier section of the backbone and at least part of the first and second partial fragment;
h. mating the first and second partial fragments based on the presence of the identifier section in the first and second amplicons, thereby identifying the mated first and second partial fragments of the DNA fragment.

77. A method for mate-pair sequencing comprising the steps of
a. providing a DNA fragment;
b. providing an backbone, the backbone comprising a first and second identifier sections and at least one first primer binding site;
c. ligating both ends of the DNA fragment with the backbone, thereby circularizing the backbone to obtain a circularized construct;
d. digesting the circularized construct with at least one enzyme to obtain a fragmented construct comprising the backbone and a first and a second partial fragment of the DNA fragment;
e. ligating adaptors containing at least one second primer binding site to the fragmented construct to obtain an adaptor-ligated fragmented construct;
f. amplifying the adaptor-ligated fragmented construct using one or more primers, thereby providing a first amplicon comprising one of the two identifier sections and the first partial fragment and a second amplicon comprising the other of the two identifier section and the second partial fragment;
g. sequencing the first and second amplicons to determine of each amplicon the nucleotide sequence of the first and second identifier section of the backbone and at least part of the first and second partial fragment;
h. mating the first and second partial fragments based on the presence of the first and second identifier sections in the first and second amplicons, thereby identifying the mated first and second fragment of the DNA fragment.

78. The method according to claim 76, wherein the DNA fragment is provided by nuclease enzyme digestion of the DNA sample, optionally using a restriction enzyme.

79. The method according to claim 76, wherein the DNA fragment is double stranded having two staggered ends, two blunt ends, or one staggered end and one blunt end.

80. The method according to claim 76, wherein the DNA fragment is size selected.

81. The method according to claim 76, wherein the backbone is double stranded having two staggered ends, two blunt ends, or one staggered and one blunt end.

82. The method according to claim 76, wherein a library of backbones is provided containing more than 2, 1000, 5000 or 10.000 backbones.

83. The method according to claim 82, wherein each backbone comprises an identifier section or a combination of identifier sections that differs from the identifier section or combination of identifier sections comprised in any other backbone in the library of backbones.

84. The method according to claim 76, wherein the fragment is ligated with a first and/or a second intermediate adaptor prior to ligation into the backbone.

85. The method according to claim 76, wherein the backbone contains an affinity tag.

86. The method according to claim 76, wherein non-circularised fragments are removed before digesting the circularized construct in step (d), optionally using exonuclease treatment or an affinity tag.

87. The method according to claim 76, wherein the enzyme in step (d) is a restriction enzyme and wherein optionally the backbone does not contain a recognition site for a restriction enzyme that is used in the digesting step (d) and/or is free of palindromic sequences of four bases or greater in length.

88. The method according to claim 76, wherein after digestion of the circularised construct in step (d), non-backbone containing fragments are removed, optionally using an affinity tag or via a capturing probe.

89. The method according to claim 76, wherein the adaptors are selected from the group consisting of a single stranded adaptor, a double stranded adaptor, and a Y-shaped adaptor.

90. The method according to claim 87, wherein the ligation of the adaptor does not restore the recognition sequence of the restriction enzyme.

91. The method according to claim 76, wherein the backbone contains primer binding sites PB Si and PBS2 and wherein two adaptors are ligated to the fragmented construct, wherein the two adaptors contain primer binding sites PBS3 and PBS4, wherein:
PBS1, PBS2, PBS3, and PBS4 are identical and the adaptor-ligated fragmented construct is amplified from one primer;
PBS1 and PBS2 are identical and PBS3 and PBS4 are identical, and the adaptor-ligated fragmented construct is amplified using two primers
PBS1 and PBS2 are identical and PBS3 and PBS4 are different, or PBS1 and PBS2 are different and PBS3 and PBS4 are identical, and the adaptor-ligated fragmented construct is amplified using three primers; or
PBS1 and PBS2 are different and PBS3 and PBS4 are different, and the adaptor-ligated fragmented construct is amplified using four primers.

92. The method according to claim 76, wherein the adaptor-ligated fragmented construct is split into a first and second subsamples, wherein the first subsample is amplified with one or more of PBS1 and PBS2 and one of PBS3 and PBS4, and wherein the second subsample is amplified with one or more of PBS1 and PBS2 and the other one of PBS3 and PBS4.

93. The method according to claim 76, wherein the sequencing is high-throughput sequencing.

94. The method according to claim 76, wherein at least one of the primers is or contains a sequencing primer and wherein optionally at least one of the primers contains an affinity probe.

95. The method according to claim 76, wherein the mating of the first and second partial fragments is based on the presence of identical identifier sections in the amplicons, or is based on non-identical identifier sections derived from the same backbone.

96. The method according to claim 76, wherein the mated pairs are used in the building of a genome scaffold.

97. The method according to claim 77, wherein a plurality of samples are used to generate genomic DNA fragments and wherein for each sample a different identifier section or a different library of identifier sections in the backbones is used such that the samples can be distinguished based on the presence of the identifier section, optionally within the primer, and wherein the identifier section or the library of identifier sections contains a sample specific identifier section.

98. The method according to claim 76, wherein the mated pairs are anchored to a physical map or to a draft genome sequence.
